(19) United States
(12) Patent Application Publication
(10) Pub. No.: US 2014/0014095 A1
(43) Pub. Date: Jan. 16, 2014

(54) TRACHEAL TUBE WITH INNER CANNULA INDICATION SYSTEM

(71) Applicant: Sean Morris, Ballymurray (IE)

(72) Inventor: Sean Morris, Ballymurray (IE)

(73) Assignee: NELLCOR PURITAN BENNETT LLC, Boulder, CO (US)

(21) Appl. No.: 13/546,082

(22) Filed: Jul. 11, 2012

(51) Int. Cl. A61M 16/04

(52) U.S. Cl. 128/200.26

(57) ABSTRACT

A tracheal tube assembly includes an outer cannula having a distal end and a proximal end, the distal end being adapted to be inserted into an airway of a patient. The assembly also includes an inner cannula adapted to be inserted into the outer cannula, a flange member disposed about the proximal end of the outer cannula, and a connector coupled to the proximal end of the outer cannula. The inner cannula and the connector form a contiguous passageway for exchanging fluid with the airway of the patient in operation. The assembly further includes an inner cannula status indication system that receives information regarding whether or not the inner cannula is an operable position with respect to the outer cannula, and, via the status indicator, provides a visual indication of whether or not the inner cannula is an operable position with respect to the outer cannula.

### BACKGROUND

The present disclosure relates generally to the field of tracheal tubes and, more particularly, to a tracheal tube having an inner cannula indication system.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A wide variety of situations exist in which artificial ventilation of a patient may be desired. For short-term ventilation or during certain surgical procedures, endotracheal tubes may be inserted through the mouth to provide oxygen and other gasses to a patient. For other applications, particularly when longer-term intubation is anticipated, tracheostomy tubes may be preferred. Tracheostomy tubes are typically inserted through an incision made in the neck of the patient and through the trachea. A resulting stoma is formed between the tracheal rings below the vocal chords. The tracheostomy tube is then inserted through the opening. In general, two procedures are common for insertion of tracheostomy tubes, including a surgical procedure and a percutaneous technique.

Such tubes may include an inner cannula, such as a reusable inner cannula, or a disposable inner cannula. The inner cannula may be disposed inside the tracheostomy tube and used as a conduit for liquids or gas being exchanged with the patient's lungs. The inner cannula may be removed for cleaning and for disposal of secretions without disturbing the placement of the tracheostomy tube. A connector is typically provided at an upper or proximal end where the tube exits the patient's airway, suitable for coupling the ventilator with the inner cannula. In some products, the inner cannula may be removed, cleaned, and reused. In other products, the inner cannula may be disposable, and a new inner cannula may then be positioned inside of the tracheal tube. By enabling the cleaning and/or replacement of the inner cannula, a ventilation circuit may be kept clean and free of secretions.

Standard connectors have been developed to allow the tracheal tube to be fluidly coupled to artificial ventilation equipment to supply the desired air or gas mixture to the patient, and to evacuate gases from the lungs. One difficulty that arises in the use of tracheal tubes in general, and tracheostomy tubes in particular, is in the connection of the tube to the ventilation equipment. For example, an inner cannula may not be installed, or may be installed improperly, possibly leading to difficulties when a connection is made to ventilation equipment.

### BRIEF DESCRIPTION OF THE DRAWINGS

Various aspects of the disclosed techniques may become apparent upon reading the following detailed description and upon reference to the drawings in which:

FIG. 1 is a perspective view of an embodiment of a tracheal tube having an inner cannula status indicator disposed in a flange member;

FIG. 2 is a perspective view of an embodiment of a tracheal tube having an inner cannula status indicator disposed in a connector;

FIG. 3 is a perspective view of an embodiment of a tracheal tube having an inner cannula status indication system coupled to a connector;

FIG. 4 is a perspective view of an embodiment of a tracheal tube having an inner cannula status indication system in communication with a ventilation system;

FIG. 5 is a schematic illustrating example components of an inner cannula status indication system; and

FIG. 6 is a flow chart illustrating an embodiment of a method through which the status of an inner cannula with respect to an outer cannula may be communicated to an operator.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described in detail below, provided herein are tracheal tube assemblies including indication systems that indicate when the inner cannula is in an operable position with respect to the outer cannula. For example, in presently contemplated embodiments, the indication system may receive information regarding whether or not the respective positions of the cannulas place the tracheal tube assembly in an operable configuration. In certain embodiments, the received information regarding the cannula positions may originate from an operator. For instance, an operator may directly input such information to the indication system, for example, by sliding a device located on a flange (or other airway component) of the assembly from one position to another. In other embodiments, however, the indication system may receive such information electronically, for example, from an associated sensor system that senses the position of the inner cannula with respect to the other cannula. Still further, in certain embodiments, the indication system may communicate whether or not the inner cannula is in an operable position with respect to the outer cannula to another system. For example, the indication system may be communicatively coupled to a ventilation system, which utilizes the relative positional information for control, for example, of the exchange of fluid between a ventilator and an airway of a patient.

The provided tracheal tube assemblies may be disposable rather than reusable and may be capable of conveying gas to and from the patient, such as during medical situations that necessitate prolonged ventilation. As such, the devices and techniques provided herein may enable maintaining a bidirectional gas flow between the patient and an external ventilation device. Accordingly, the tracheostomy tube assemblies provided herein may be adapted to be inserted into the trachea via a surgical incision in the neck such that after insertion of the tube into the trachea, a portion of the tube remains outside the patient. This portion extends outwards from the neck and may connect the tracheostomy tube to a ventilator or other medical device. That is, the provided tracheostomy tube assemblies may be used in conjunction with auxiliary devices, such as airway accessories, ventilators, humidifiers, and so forth, which may cooperate with the tube assemblies to maintain airflow to and from the lungs of the patient. For example, the tracheal tubes may be coupled to an adapter or connector that is configured to couple the tracheostomy tube assemblies described herein to the desired auxiliary device.

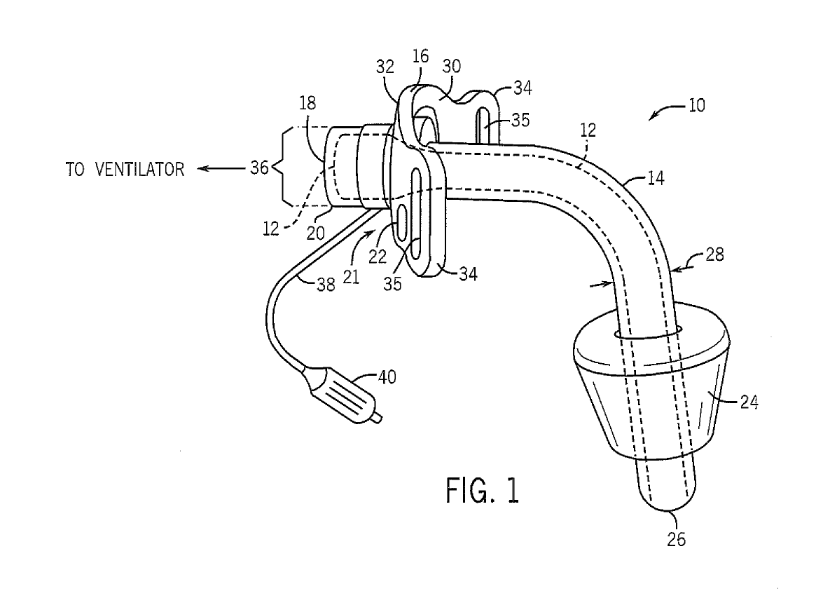

Turning now to the drawings, a tracheal tube consistent with one embodiment is illustrated in FIG. 1. As shown, a tracheal tube assembly 10 represented in the figures is a tracheostomy tube, although aspects of this disclosure could be applied to other airway devices or structures utilizing a dual or multiple cannula configuration. The application to a tracheostomy tube is apt, however, insomuch as such tubes tend to be worn for longer periods of time, and thus may include a removable and/or disposable inner cannula 12 shown disposed inside of an outer cannula 14, which may be useful in maintaining a clean ventilation circuit.

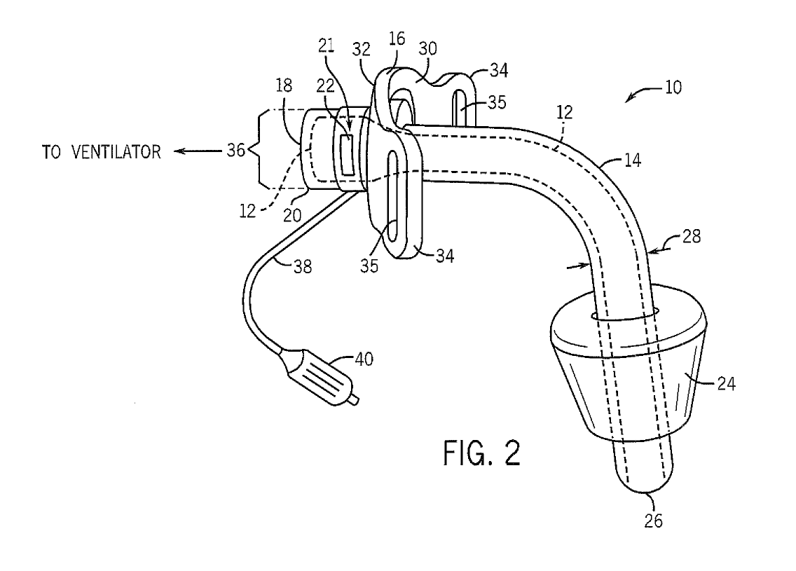

The outer cannula 14 is illustrated extending both distally as well as proximally from a flange member 16. The inner cannula 12 may be introduced through an opening 18 of an end connector 20 inside of the outer cannula 14. In the depicted embodiment, an inner cannula status indication system 21 having a status indicator 22 is provided. In the embodiment of FIG. 1, the status indicator 22 is shown positioned on the flange member 16 to facilitate visual inspection by a clinician when the tube assembly 10 is positioned within a patient. However, it should be noted that in other embodiments, the status indicator 22 and other components of the indication system 21 may be located in other desirable locations. For example, as shown in FIG. 2, the status indicator 22 may be located on the connector 20, or at any other desired location on any suitable component of the breathing circuit.

Regardless of its location, however, the status indicator 22 may enable a clinician or other user to easily identify whether the inner cannula 12 is properly placed with respect to the outer cannula 14. The foregoing feature may offer advantages over existing systems because the operator may easily identify that the cannula assembly is in an operable configuration, thus ensuring proper placement of the inner cannula 12 during use.

In the embodiments illustrated in FIGS. 1 and 2, the status indicator 22 is shown as a rectangular or ovular piece. In other embodiments, however, the status indicator 22 may be provided in other shapes, such as a circle, a square, a triangle, a parallelogram, a hexagon, and so forth, or other configurations, such as an annular ring disposed about the connector 20 and/or the cannula 14. Additionally, more than one status indicator 22 may be used in some embodiments. For example, in one embodiment, a first status indicator 22 may be provided on the end connector, and a second status indicator 22 may be provided on the flange member. The foregoing placement may enable the presence and positioning of the inner cannula 12 to be easily identified during ventilation operations by a number of different users positioned at a variety of locations about the intubated patient.

Still further, it should be noted that the status indicator 22 may be capable of conveying at least one parameter of the position of the inner cannula 12 with respect to the outer cannula 14 through a variety of suitable devices and systems. For example, in one embodiment, the status indicator 22 may be color coded, with each color indicating a different relative position of the cannulas with respect to one another. In such an embodiment, a first color (e.g., grey) may indicate that the tracheal tube assembly is not configured to receive an inner cannula, a second color (e.g., green) may indicate that the tracheal tube assembly is configured to receive an inner cannula and the cannulas are positioned in an operable manner with respect to one another, and a third color (e.g., red) may indicate that the tracheal tube assembly is configured to receive an inner cannula but the cannulas are not positioned in an operable manner with respect to one another. Still further, in other embodiments, the status indicator 22 may be an electronic display configured to communicate the respective position of the inner cannula to the user, for example, via visual or audio cues. Indeed, the forms that the status indicator 22 may take are specific to the application and are subject to a variety of implementation-specific considerations. The embodiments disclosed herein are merely examples.

During intubation, the tracheal tube assembly 10 is placed through an opening formed in the neck and trachea of a patient, and extending into the patient's airway. The embodiment illustrated in the figures includes a sealing cuff 24, although in practice a wide range of tube designs may be used, including tubes having no cuffs or tubes having multiple cuffs around the outer cannula 14. The inner cannula 12 in the illustrated embodiment Rains a conduit from which liquids or gases, including medications, may enter through the proximal opening 18 and exit through a distal opening 26. The cannula has an outer dimension 28 allowing it to fit easily through an incision made in the neck and trachea of the patient. In practice, a range of such tubes may be provided to accommodate the different contours and sizes of patients and airways. Such tube families may include tubes designed for neonatal and pediatric patients, as well as for adults. By way of example only, outer dimension 28 of the tube 20 may range from approximately 4 mm to approximately 16 mm in some embodiments.

In one embodiment, the outer cannula 14 enters the flange member 16 along a lower face 30 and protrudes through an upper face 32 of the flange member 16. When in use, the face 30 will generally be positioned against the neck of a patient, with the cannula extending through an opening formed in the neck and trachea. A pair of side wings or flanges 34 extend laterally and enable a strap or retaining member (not shown) to hold the tube assembly in place on the patient. In the illustrated embodiment, apertures 35 are formed in each side flange 34 to allow the passage of such a retaining device. In many applications, the flange member 16 may be taped or sutured in place as well.

In some embodiments, the outer cannula 14 may also include a suction lumen (not shown in FIG. 1) that extends from a location on the proximal end of the outer cannula 14 positioned outside the body when in use to a location around the cuff 24 inside the body. The suction lumen may terminate in a port through which secretions accumulated around the cuff may be aspirated. For example, a port may be located above the cuff 24 or one or more ports may be located anywhere along the length of the outer cannula 14 such that they aspirate secretions from the airway of the patient. Further, in some embodiments, an exterior suction tube may connect to the suction lumen for the removal of the suctioned fluids, for example, via a vacuum connected to the exterior suction tube.

The end connector 20 is formed in accordance with industry standards to permit and facilitate connection to a ventilation system. By way of example, standard outer dimensions may be provided as indicated at reference numeral 36 that enable a mating connector piece to be secured on the connector shown. By way of example, a presently contemplated standard dimension 36 accommodates a 15 mm connector, although other sizes and connector styles may be used. In use, then, air or other gas may be supplied through the connector and the inner cannula 12, and gases may be extracted from the patient. For example, the tube assembly 10 may be inserted into the patient's airways, and the cuff 24 may then be inflated through an inflation lumen 38. In some embodiments, a pilot balloon 40 may then indicate that air is in the cuff 24, thus sealing the patient's airway. However, in other embodiments, the pilot balloon 40 may be eliminated, or the pilot balloon 40 may be utilized as one indication device, which is relied upon in part in combination with other indicators to determine when air is in the cuff 24. Once the tracheal tube is positioned and secured, a ventilator may be coupled to the end connector 20, as described in more detail below. By providing the status indicator 22 (or any of the variety of other indicators and indication systems described below), the tube assembly 10 may enable a more efficient ventilation circuit and reduce or prevent the likelihood of certain errors, such as ventilating the tube assembly 10 when the assembly is missing the inner cannula 12.

As mentioned above, the tracheal tube assembly 10 of FIGS. 1 and 2 may, in some embodiments, be connected to a ventilator via a ventilation tube, thus enabling an assisted airway circuit through the patient's lungs. Additionally, it should be noted that the tracheal tube assembly 10 may be connected to other medical devices, such as a suction device, a T-junction, a medicine delivery system, and so forth, as desired according to implementation-specific considerations. Indeed, the end connector 20 may enable the attachment of one or more medical devices to the tracheal tube assembly 10. When such devices are attached to the assembly 10 in this manner, the status indicator 22 may be used to verify the presence and proper placement of the inner cannula 12, thus providing confirmation that the tube assembly 10 has been properly assembled into an operable configuration.

Additionally, as appreciated by one skilled in the art, it should be noted that the components of the tracheal tube assembly 10 may be formed from various suitable materials via any appropriate manufacturing process. For example, the end connector 20 may be manufactured of materials such as a polyvinyl chloride (PVC), a PEBAX silicone, a polyurethane, thermoplastic elastomers, a polycarbonate plastic, a silicon, or an acrylonitrile butadiene styrene (ABS). For further example, the inner and/or outer cannulas 12 and 14 may be formed from a soft polyvinyl chloride (PVC) or another suitable plastic (e.g., polyurethane, thermoplastic elastomers, etc.) through an extrusion process.

Still further, in certain embodiment, the end connector 20 and/or of the flange member 16 may be molded, overmolded, computer numerical control (CNC) machined, milled, or otherwise formed into the desired shape. In one embodiment, such as that which is shown in FIG. 2, a mold or machine used to manufacture the end connector 20 may produce the end connector 20 having a slot sized to fit the status indicator 22. The status indicator 22 (and any other desired components of the status indication system 21) may then be added to the end connector 20, for example, by overmolding, gluing, thermally bonding, or more generally, attaching the status indicator 22 to the end connector 20.

In certain embodiments, the illustrated components may be provided as a tube assembly kit instead of in an assembled form. In such embodiments, the user or clinician may perform final assembly of the tracheal tube 10 by selecting a desired inner cannula 12 and then inserting the inner cannula 12 into the outer cannula 14 prior to intubation of the patient. More specifically, a distal end of the inner cannula 12 may be manually inserted inwardly into the outer cannula 14 through the opening 18. If the inner cannula 12 has been properly inserted, the status indicator 22 may activate a display, or otherwise communicate to the user that the inner cannula 12 is now in place with respect to the outer cannula 14. Once assembled in this manner, the tracheal tube 10 may then be inserted into the patient's trachea, and, during ventilation activities, the clinician may easily verify the presence of the inner cannula 12, as well as the position of the inner cannula 12 relative to the outer cannula 14.

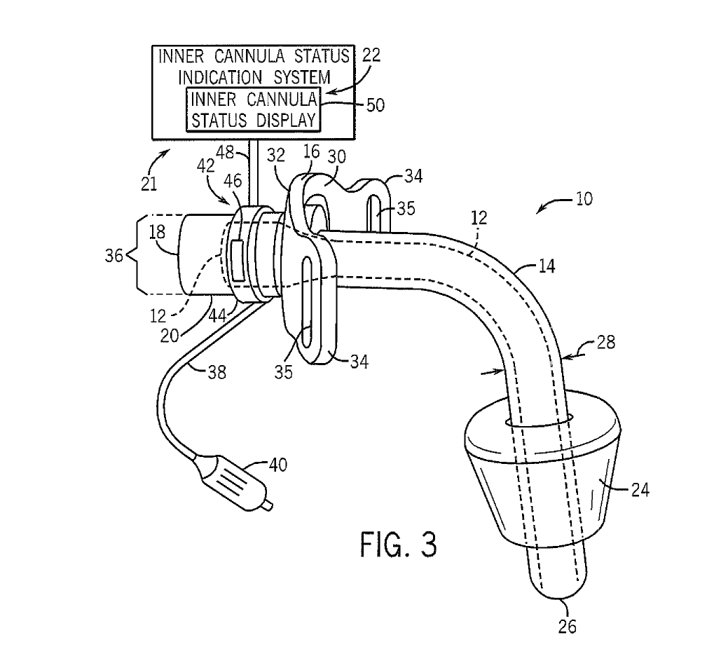

FIG. 3 illustrates an alternate embodiment of the tracheal tube assembly 10 of FIGS. 1 and 2 having the inner cannula status indication system 21 mounted on the connector 20. In this embodiment, the indication system 21 includes a collar assembly 42 including a collar 44 with a sensor 46 mounted thereon and a coupling device 48 that connects the collar 44 to an inner cannula status display 50. That is, in this embodiment, the sensor 46 that is configured to sense the position of the inner cannula 12 with respect to the outer cannula 14 is attached to the tracheal tube assembly 10, but the display 50 that communicates the status of the inner cannula 12 to a clinician is not located directly on the assembly 10. The foregoing feature may enable the display 50 to be larger in size or more detailed in the amount of data that is communicated to the caregiver, since the dimensions of the display 50 are not constrained by the area available on the tracheal tube components. However, the sensor 46 may still remain in close proximity to the inner and outer cannulas 12 and 14 to determine their relative positions. Additionally, it should be noted that the sensor 46 may be configured in a variety of different ways, depending on implementation-specific considerations. For example, in one embodiment, the sensor 46 may be configured as an annular ring disposed about the circumference of the collar 44 and the connector 20.

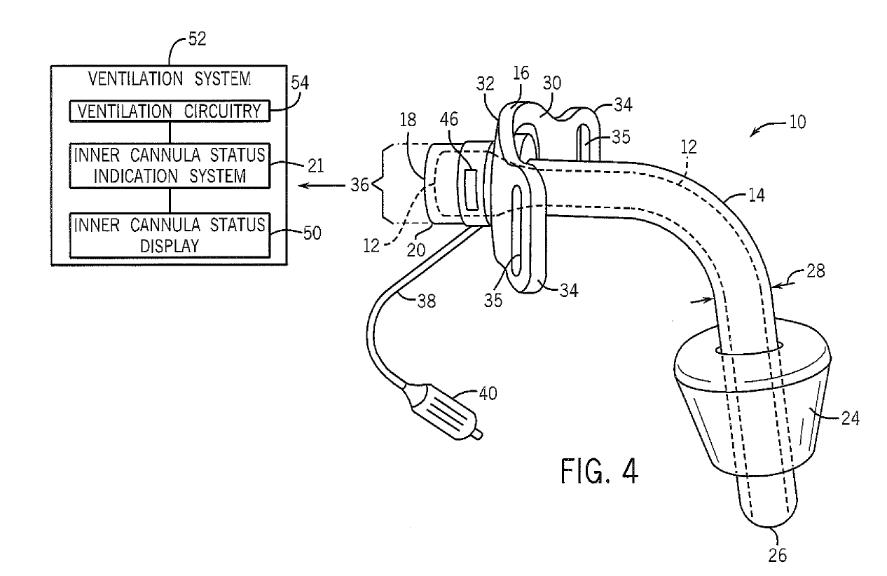

FIG. 4 illustrates another embodiment of the tracheal tube assembly 10 in which the positional sensor 46 senses the position of the inner cannula 12 with respect to the outer cannula 14 and communicates the sensed position to the indication system 21 disposed in a ventilation system 52. Such communication may be wireless or wired, depending on the given application. Once the relative position of the inner cannula 12 with respect to the outer cannula 14 is communicated to the indication system 21, the indication system may communicate whether or not the cannula assembly is in an operable or inoperable position to one or both of an operator (via the display 50) and ventilation circuitry 54 (e.g., a ventilator). The foregoing information may subsequently be utilized for control and/or operation of the assembly 10. Further, it should be noted that in certain embodiments, data corresponding to the relative position of the inner cannula 12 with respect to the outer cannula 14 may be communicated wirelessly (or via a wired connection, if available and desired) to accessory devices, such as a smart phone, smart watch, etc., for example, for remote monitoring.

For instance, in one embodiment, the indication system 21 may alert the ventilation circuitry 54 when the cannulas are positioned in an operative position, and the ventilation system 52 may then begin the exchange of fluid from the ventilation circuitry 54 with the airway of the patient through a contiguous passageway formed by the properly placed inner cannula 12 and the connector 20. Additionally, if desired in the given application, the indication system 21 may also display the operable status of the tube assembly to the operator via the display 50. Still further, in instances in which the positional sensing system 46 indicates that the cannulas are in an inoperable position, the ventilation system 52 may alert the operator to the presence of an error and/or may cease the exchange of fluid between the ventilator and the patient, again depending on implementation-specific considerations. Indeed, the relative position of the inner cannula 12 with respect to the outer cannula 14 may be utilized in a variety of downstream control schemes or algorithms to aid in the operation of the overall system if desired.

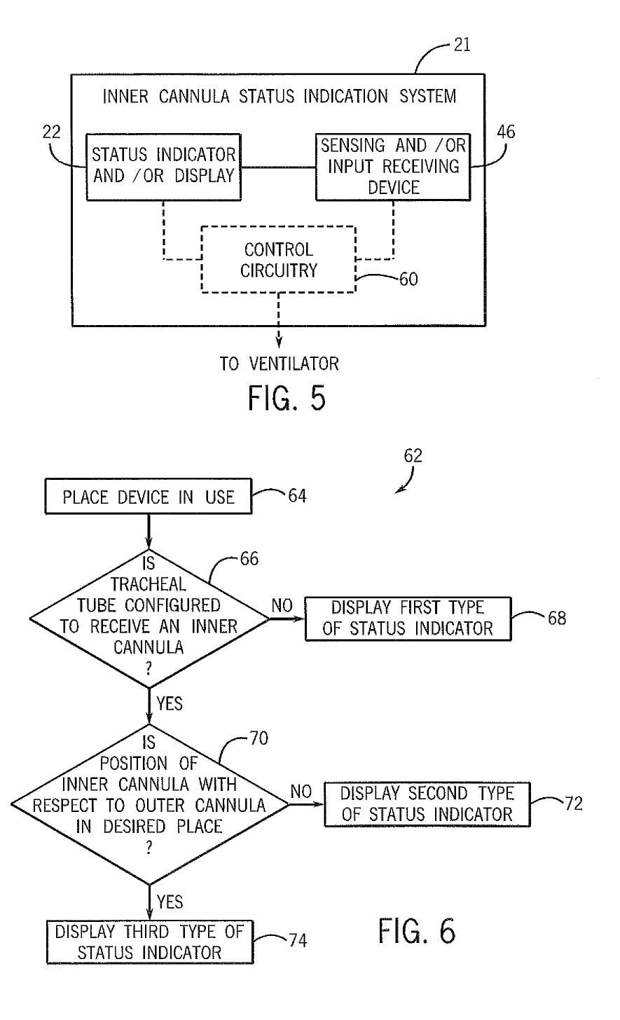

FIG. 5 is a block diagram illustrating examples of components that may be included in one embodiment of the inner cannula status indication system 21. In the illustrated embodiment, the system 21 includes the status indicator 22, which may be a small device mounted on the tracheal tube assembly or a larger display associated with an auxiliary device. Further, the indication system 21 also includes the sensing device 46. In one embodiment, the sensing device 46 may take the form of a device that is configured to receive an input from an operator regarding the relative position of the inner cannula 12 to the outer cannula 14. For example, such an embodiment may enable an operator to insert the inner cannula 12 into the outer cannula 14 and to reposition, for example, a sliding indicator, thus indicating to another downstream user that the inner cannula 12 has been properly placed in the outer cannula 14.

Still further, it should be noted that in some embodiments, the indication system 21 may be completely or partially automated, and in such embodiments, control circuitry 60 may be provided with or without associated memory, energy storage device(s), and so forth. In other embodiments, however, the indication system 21 may be configured as a manual system in which a user inputs the information regarding the position of the cannulas and makes a mechanical adjustment that communicates that information to a future user. In such a system, the control circuitry 60 and any other electronics may be eliminated.

FIG. 6 is a flow chart illustrating an embodiment of a method 62 through which the status of the inner cannula 12 with respect to an outer cannula 14 may be communicated to an operator. The method 62 begins by placing the device in use (block 64) and inquiring as to whether the tracheal tube is configured to receive an inner cannula (block 66). That is, a check is performed to identify whether the given tracheal tube assembly is designed to receive an inner cannula, or whether it is not a dual cannula system. If the assembly is not designed to receive an inner cannula, a first type of status indicator is displayed (block 68). For example, a first color, such as grey, may be displayed to the operator to communicate to the operator that the assembly is not typically associated with an inner cannula.

The method 62 proceeds with an inquiry as to whether the position of the inner cannula 12 with respect to the outer cannula 14 is in the desired place (block 70). That is, a check may be performed to identify whether the cannulas are positioned in an operable or inoperable position, for example, within a preset positional tolerance. If the cannulas are not in an operable position, a second type of status indicator is displayed (block 72). For example, a second color, such as red, may be displayed to the operator to communicate to the operator that the assembly is not ready for operation. However, if the cannulas are in an operable position, a third type of status indicator (e.g., a green color) is displayed (block 74). In this way, the status of the inner cannula may be communicated to the clinician, or other end user.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A tracheal tube assembly, comprising:
an outer cannula comprising a distal end and a proximal end, the distal end being configured to be inserted into an airway of a patient;
an inner cannula configured to be inserted into the outer cannula;
a flange member disposed about the proximal end of the outer cannula;
a connector coupled to the proximal end of the outer cannula, wherein the inner cannula and the connector form a contiguous passageway for exchanging fluid with the airway of the patient in operation; and
an inner cannula status indication system comprising a status indicator that indicates at least one parameter of the position of the inner cannula with respect to the outer cannula in operation.

2. The assembly of claim 1, wherein the at least one parameter of the position of the inner cannula comprises an alignment between a portion of the inner cannula and a corresponding portion of the outer cannula.

3. The assembly of claim 1, wherein the status indicator is positioned on the flange member and configured to display a color corresponding to the relative position of the inner cannula with respect to the outer cannula.

4. The assembly of claim 3, wherein the status indicator is configured to display a red color to indicate that the tracheal tube assembly is configured to include an inner cannula, but the inner cannula is not present in the outer cannula.

5. The assembly of claim 3, wherein the status indicator is configured to display a green color to indicate that the tracheal tube assembly is configured to include an inner cannula, and the inner cannula is present in the outer cannula.

6. The assembly of claim 1, wherein the inner cannula status indication system comprises a sensor system configured to sense the position of the inner cannula with respect to the outer cannula.

7. The assembly of claim 1, wherein the inner cannula status indication system comprises a user interface configured to enable an operator to input the at least one parameter of the position of the inner cannula into the inner cannula status indication system.

8. The assembly of claim 1, comprising a conduit coupled to the connector for connecting to a at least one of an airway accessory, a ventilator, a humidifier, or a combination thereof.

9. The assembly of claim 1, comprising a cuff disposed about the distal end of the outer cannula and configured to be inflated to seal against walls of the airway of the patient.

10. The assembly of claim 1, wherein the flange member comprises a pair of apertures configured to receive securement straps adapted to secure the tracheal tube assembly to the patient's neck.

11. A tracheal tube system, comprising:
a tracheal tube assembly, comprising:
an outer cannula comprising a distal end and a proximal end, the distal end being configured to be inserted into an airway of a patient;
an inner cannula configured to be inserted into the outer cannula; and
a flange member disposed about the proximal end of the outer cannula;
a ventilation system coupled to the proximal end of the outer cannula via a connector, wherein in operation, the ventilation system exchanges fluid with the airway of the patient through a contiguous passageway formed by the inner cannula and the connector;
a positional sensing system configured to sense a position of the inner cannula with respect to the outer cannula; and
an inner cannula status indication system communicatively coupled to the ventilation system and to the positional sensing system, wherein the inner cannula status indication system is configured to receive data corresponding to the relative position of the inner cannula with respect to the outer cannula from the positional sensing system, and to alert the ventilation system when the inner cannula is in an operable position with respect to the outer cannula.

12. The system of claim 11, wherein the inner cannula status indication system comprises a status indicator configured to indicate the relative position of the inner cannula with respect to the outer cannula to an operator.

13. The system of claim 12, wherein the status indicator comprises a window disposed in the flange member and configured to visually indicate to an operator whether or not the inner cannula is in the operable position with respect to the outer cannula.

14. The system of claim 13, wherein the window comprises a rectangular shape, a swine shape, a circular shape, a parallelogram shape, or a combination thereof.

15. The system of claim 12, wherein the status indicator comprises a window disposed in the connector and configured to visually indicate to an operator whether or not the inner cannula is in the operable position with respect to the outer cannula.

16. The system of claim 15, wherein the window comprises an annular window disposed about the circumference of the connector.

17. The system of claim 11, wherein the ventilation system is configured to alert an operator when the operator indicates that ventilation is desired and the inner cannula is not in an operable position with respect to the outer cannula.

18. A tracheal tube assembly, comprising:
an outer cannula comprising a distal end and a proximal end, the distal end being configured to be inserted into an airway of a patient;
an inner cannula configured to be inserted into the outer cannula;
a flange member disposed about the proximal end of the outer cannula;
a connector coupled to the proximal end of the outer cannula, wherein the inner cannula and the connector form a contiguous passageway for exchanging fluid with the airway of the patient in operation; and
an inner cannula status indication system comprising a status indicator, wherein, during operation, the inner cannula status indication system receives information regarding whether or not the inner cannula is an operable position with respect to the outer cannula, and, via the status indicator, provides a visual indication of whether or not the inner cannula is an operable position with respect to the outer cannula.

19. The assembly of claim 18, wherein the inner cannula status indication system receives information regarding whether or not the inner cannula is an operable position with respect to the outer cannula via operator input.

20. The assembly of claim 18, wherein the inner cannula status indication system receives information regarding whether or not the inner cannula is an operable position with respect to the outer cannula from a sensing system configured to sense the position of the inner cannula.
